(19) United States
(12) Patent Application Publication
(10) Pub. No.: US 2016/0074054 A1
(43) Pub. Date: Mar. 17, 2016

(54) IRIS RETRACTOR FORCEPS

(71) Applicant: APX OPHTHALMOLOGY LTD., Haifa (IL)

(72) Inventors: Ehud ASSIA, Tel Aviv (IL); Eliahu ELIACHAR, Haifa (IL); Nir LILACH, Kfar Yehoshua (IL)

(73) Assignee: APX OPHTHALMOLOGY LTD., Haifa (IL)

(21) Appl. No.: 14/784,255

(22) PCT Filed: Apr. 18, 2014
(86) PCT No.: PCT/IL2014/050367

§ 371 Date: Oct. 13, 2015

Related U.S. Application Data
(60) Provisional application No. 61/813,658, filed on Apr. 19, 2013.

(51) Int. Cl. A61B 17/28

(52) U.S. Cl. CPC A61B 17/28

(57) ABSTRACT

An assembly comprising: an iris retractor forceps comprising two forceps legs which join together at a proximal junction, the forceps legs comprising distal graspers for grasping an iris retractor or a holder for such an iris retractor; and a locking element slidingly received in a groove in the forceps legs, the locking element sliding between at least two positions, the at least two positions comprising: a first position in which the forceps legs are spread apart, and a second position in which the forceps legs are squeezed together, wherein in the second position the locking element is held in a locked position.

### FIELD OF THE INVENTION

The present invention relates generally to iris retractor assemblies used in ophthalmic surgical procedures, and particularly to a forceps for grasping an iris retractor.

### BACKGROUND OF THE INVENTION

There are various ophthalmic procedures that require the dilation of the pupil. For example, a lens with a cataract is typically removed from the eye by phacoemulsification. This procedure breaks up the lens typically with an ultrasonically driven tool. The tool has an aspiration port that aspirates the broken lens material from the patient's ocular-chamber. It is desirable to extend the pupil during phacoemulsification to provide the surgeon with a wide view of the lens. One technique for extending the pupil includes pulling back or retracting the iris with what is referred to as an iris retractor, and holding the iris at its outer edges.

PCT Patent Application WO/2011/053945 (PCT/US2010/055026) of the present assignee describes an iris retractor that has iris grabbing hooks disposed or formed at a distal end of slender elements. A proximal handle is at a proximal end of the slender elements. The slender elements rigidily or resiliently move between retracted and expanded positions by manipulation of the slender elements. In the retracted position, the hooks are close to one another and the slender elements are close to one another. In the expanded position, the hooks are separate and spaced apart from each other and the slender elements are separate and spaced apart from each other. The surgeon inserts the slender elements in the retracted position through a small incision near the limbus of the eye, manipulates the handle to move the slender elements to the expanded position, and grasps and retracts a portion of the iris with the hooks. The incisions for the insertion of the slender elements is made at different positions than the incision for phacoemulsification.

PCT Patent Application PCT/US2012/061513 of the present assignee describes further iris retractor assemblies. In one example, pincer interface members are located at proximal ends of the slender elements. The pincer interface members include posts that jut out of the proximal ends perpendicular to a plane of the slender elements and parallel to a pivot axis of the pivot member.

The foregoing examples of the related art and limitations related therewith are intended to be illustrative and not exclusive. Other limitations of the related art will become apparent to those of skill in the art upon a reading of the specification and a study of the figures.

### SUMMARY OF THE INVENTION

The following embodiments and aspects thereof are described and illustrated in conjunction with systems, tools and methods which are meant to be exemplary and illustrative, not limiting in scope.

In one embodiment,the invention provides an assembly comprising: an iris retractor forceps comprising two forceps legs which join together at a proximal junction, the forceps legs comprising distal graspers for grasping an iris retractor or a holder for an iris retractor; and a locking element slidingly received in an elongated channel (or alternatively at least two grooves: distal and proximal) formed in the forceps legs, the locking element sliding between at least two positions, the at least two positions comprising: (a) a first position in which the forceps legs are spread apart, and (b) a second position in which the forceps legs are squeezed together, wherein in the second position the locking element is held in a locked position.

In one embodiment, the invention further provides a kit comprising: one or more iris retractor assemblies; a holder comprising one or more iris retractor assemblies or holding assemblies (the holding assemblies are configured to hold one or more iris retractor assemblies); an iris retractor forceps comprising two forceps legs which join together at a proximal junction, the forceps legs comprising distal graspers for grasping the holder; and a locking element slidingly received in an elongated channel (or alternatively at least two grooves) formed in the forceps legs, the locking element sliding between at least two positions, the at least two positions comprising: (a) a first position in which the forceps legs are spread apart, and (b) a second position in which the forceps legs are squeezed together, wherein in the second position the locking element is held in a locked position.

In one embodiment, the invention further provides a kit comprising: one or more iris retractor assemblies; an iris retractor forceps comprising two forceps legs which join together at a proximal junction, the forceps legs comprising distal curved grasping tips for grasping an iris retractor assembly of the one or more iris retractor assemblies; and a locking element slidingly received in an elongated channel (or alternatively at least two grooves) formed in the forceps legs, the locking element sliding between at least two positions, the at least two positions comprising: (a) a first position in which the forceps legs are spread apart, and (b) a second position in which the forceps legs are squeezed together,wherein in the second position the locking element is held in a locked position. In one embodiment, at least grooves define a first position in which the forceps legs are spread apart, and a second position in which the forceps legs are squeezed together.

In addition to the exemplary aspects and embodiments described above, further aspects and embodiments will become apparent by reference to the figures and by study of the following detailed description.

### BRIEF DESCRIPTION OF THE DRAWINGS

Exemplary embodiments are illustrated in referenced figures. Dimensions of components and features shown in the figures are generally chosen for convenience and clarity of presentation and are not necessarily shown to scale. The figures are listed below.

FIG. 1A is a simplified, perspective illustration of an iris retractor forceps, constructed and operative in accordance with an embodiment;

FIGS. 1B, 1C and 1D are exploded illustrations of an iris retractor forceps, constructed and operative in accordance with an embodiment;

FIGS. 2A, 2B and 2C are simplified side, front and top view illustrations, respectively, of the iris retractor forceps of FIG. 1A in an expanded (relaxed, non-gripping) orientation;

FIGS. 3A, 3B, 3C and 3D are simplified side, front and top view illustrations, respectively, of the iris retractor forceps of FIG. 1A in a contracted (squeezed, gripping) orientation, with a locking element slid into a locked position and fingers pressed illustration;

FIGS. 4A, 4B, 4C and 4D are simplified perspective, perspective enlarged, top-view and top-view enlarged illustrations, respectively, of the iris retractor forceps of FIG. 1A placed adjacent to the holder positioned to attach to the Iris Retractor when contracted/squeezed, in accordance with an embodiment;

FIG. 5 is an illustrations of simplified perspective view of an exemplary iris retractor assembly in an expanded orientation, in accordance with an embodiment;

FIGS. 6A and 6B are illustrations of simplified perspective and perspective enlarged views of an exemplary Iris retractor forceps in an expanded orientation and the iris retractor assembly of FIG. 5;

FIGS. 7A and 7B are illustrations of simplified perspective and perspective enlarged views of the iris retractor forceps of FIGS. 6A and 6B in a contracted orientation holding the iris retractor assembly of FIG. 5 in a contracted orientation using the locking element slid into a locked position;

FIGS. 7C and 7D are illustrations of simplified perspective and perspective enlarged views of the iris retractor forceps of FIGS. 6A and 6B in a contracted orientation holding the iris retractor assembly of FIG. 5 in a contracted orientation using fingers force;

FIG. 8A is an illustration of a top view of the iris retractor assembly of FIG. 5 in an extended orientation, indicating dimensions of the iris retractor assembly, as described herein below;

FIG. 8B is an illustration of a side view of the iris retractor assembly of FIG. 8A, indicating dimensions of the iris retractor assembly, as described herein below;

FIG. 9A is an illustration of a top view of the iris retractor of FIG. 5, indicating dimensions of the iris retractor, as described herein below;

FIG. 9B is an illustration of an enlarged side view of the indicated portion of the iris retractor of FIG. 5, indicating dimensions of the iris retractor, as described herein below;

FIGS. 10A and 10B are illustrations of a side view of the iris retractor forceps of FIG. 1A and an enlarged side view of the indicated portion of the iris retractor forceps of FIG. 10A, indicating dimensions of the iris retractor forceps, as described herein below; and

FIGS. 10C and 10D are illustrations of a top view of the iris retractor forceps of FIG. 1A and an enlarged top view of the indicated portion of the iris retractor forceps of FIG. 10C, indicating dimensions of the iris retractor forceps, as described herein below.

### DETAILED DESCRIPTION OF EMBODIMENTS

Kits, assemblies and forceps devices for ophthalmic surgical procedures are disclosed herein. Iris retractor forceps (or simply “forceps”) for grasping and holding iris retractor assemblies or a holder for such iris retractor assemblies, as described in more detail herein below, may be used with a locking element to hold an iris retractor assembly (or simply “iris retractor” or “retractor”). The locking element may enable the forceps to hold a loaded iris retractor, such as when the retractor is not yet in use or when passing the retractor from one person to another. The locking element may, in some embodiments, lock the iris retractor to the forceps. For example, loaded forceps may be left loaded and locked on the sterile table for the physician's use.

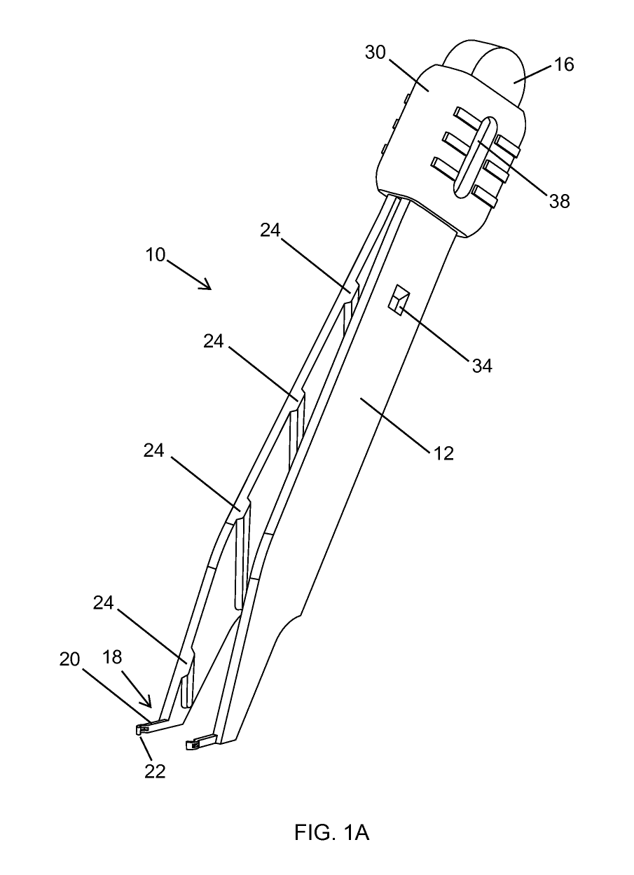

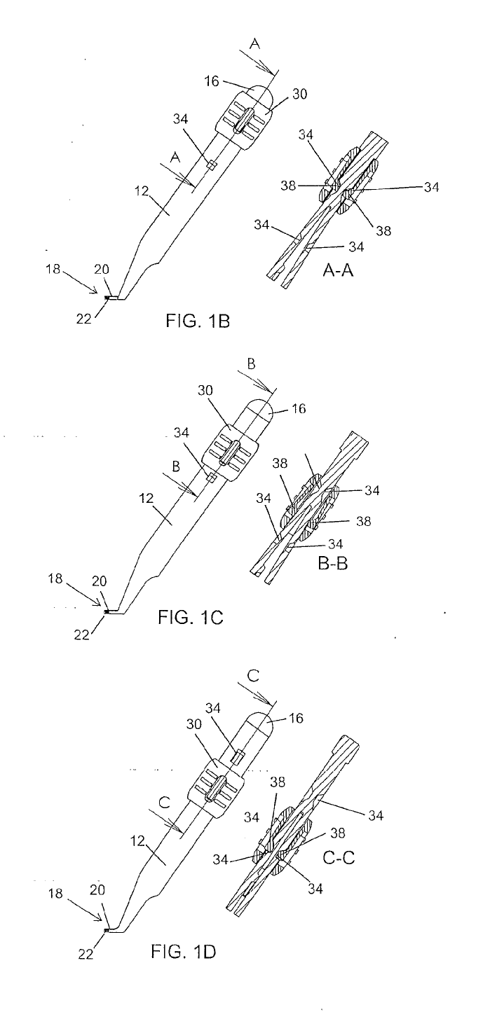

Reference is now made to FIG. 1A, which illustrates an iris retractor forceps 10, constructed and operative in accordance with a non-limiting embodiment. Iris retractor forceps 10 is designated for grasping and holding iris retractor assemblies or a holder for such iris retractor assemblies. The iris retractor assemblies may include iris retractor assemblies as disclosed in PCT Patent Application Publication WO 2013/062983, which is hereby incorporated by reference in its entirety.

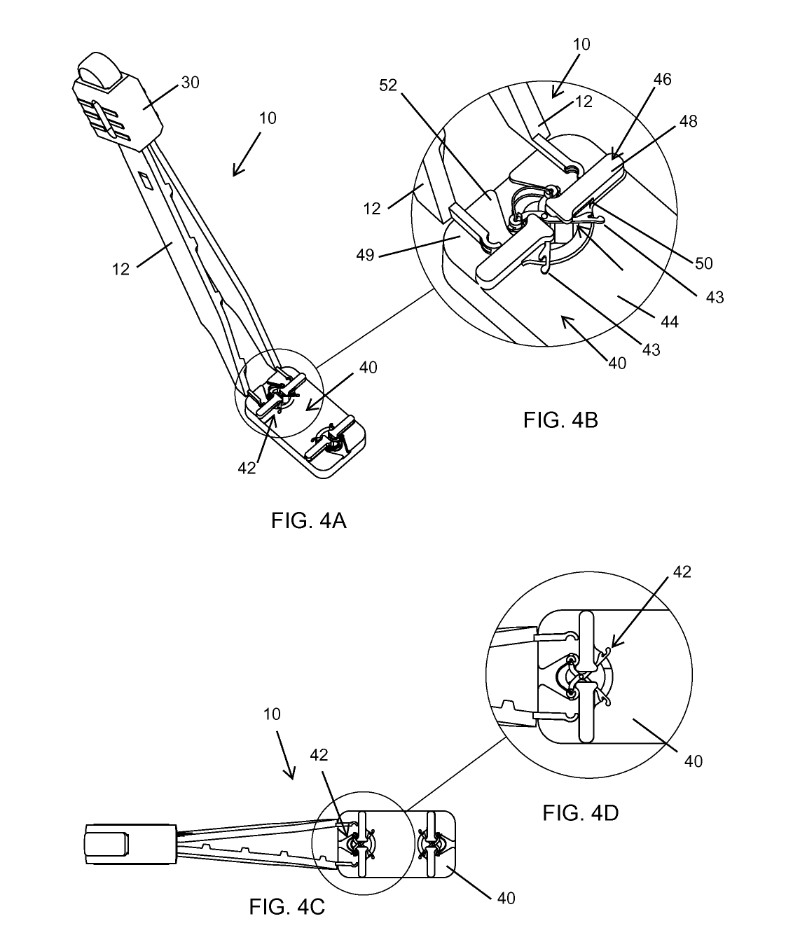

Iris retractor forceps 10 includes two forceps legs 12 which join together at a proximal junction 14 (FIGS. 1B, 1C, 1D). A knob or a handle member 16 may be provided near the proximal junction 14. Distal graspers 18 of the forceps legs 12 are constructed to facilitate grasping an iris retractor or a holder for such an iris retractor (shown in FIGS. 4A-4D). For example, distal graspers 18 of the forceps legs 12 may have a protrusion 20 that angularly extends from the rest of the leg 12. Protrusion 20 may have a distal, curved grasping tip 22. One or more stoppers 24 may be provided on the inner surfaces of legs 12. In some embodiments, iris retractor forceps comprise 1 to 7 stoppers 24. In another embodiment, iris retractor forceps comprise 2 to 6 stoppers 24. In another embodiment, iris retractor forceps comprise 3 to 5 stoppers 24. The stoppers 24 prevent over-squeezing of the forceps legs 12, preventing possible excessive force from being applied to the iris retractor assembly and preventing possible mechanical distortion.

Each of the forceps legs 12 may be formed with grooves 32 and/or 34 one proximal and one distal, into which a flexible member 38 of locking element 30 may be slidingly received. In another embodiment, elongated channel may be formed with two or more grooves, for example grooves 32 and 34, one proximal and one distal. In one embodiment, no elongated channel is present and at least grooves (one proximal and one distal) define a first position in which the forceps legs are spread apart, and a second position in which the forceps legs are squeezed together.

In another embodiment, a locking element is slidingly received in a groove formed in the forceps legs, wherein the locking element is sliding between at least two positions: a distal position and a proximal position. In another embodiment, a locking element is slidingly received in a distal groove or a proximal groove formed in the forceps legs, wherein the locking element is slides between a distal position defined by a distal groove and a proximal position defined by a proximal groove. In some embodiments, the phrase “a distal groove and a proximal groove formed in the forceps legs” includes parallel distal grooves in each forceps leg and parallel proximal grooves in each forceps leg. In some embodiments, the phrase “a distal groove and a proximal groove formed in the forceps legs” includes a single distal groove in one of the forceps leg and a single proximal groove in the same leg. In some embodiments, the phrase “a distal groove and a proximal groove formed in the forceps legs” includes a distal groove and a proximal groove in one leg. In some embodiments, the phrase “a distal groove and a proximal groove formed in the forceps legs” includes a distal groove and a proximal groove formed in each one of the forceps leg.

In another embodiment, locking element 30 may include two pieces which mate with each other when assembled in a groove. In another embodiment, locking element 30 may include an inner rib 36 with a bulging flexible member 38. The flexible member 38 locks (“clicks”) into grooves 32 or 34.

Referring to FIGS. 1B, 1C, 1D, iris retractor forceps 10 is shown here in three locking element 30 positions, expanded, partially contracted and contracted.

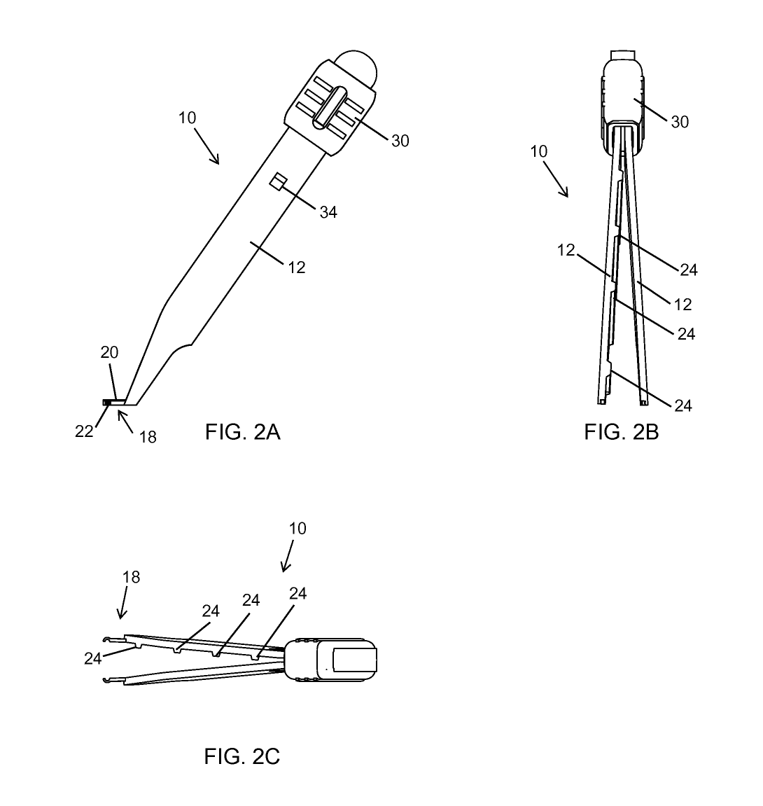

Referring to FIGS. 2A-2C, iris retractor forceps 10 is in an expanded (relaxed, non-gripping) orientation, in which the forceps legs 12 are spread apart. The flexible member 38 of locking element 30 is locked into the proximal groove 32 (not seen in FIGS. 2A-2C).

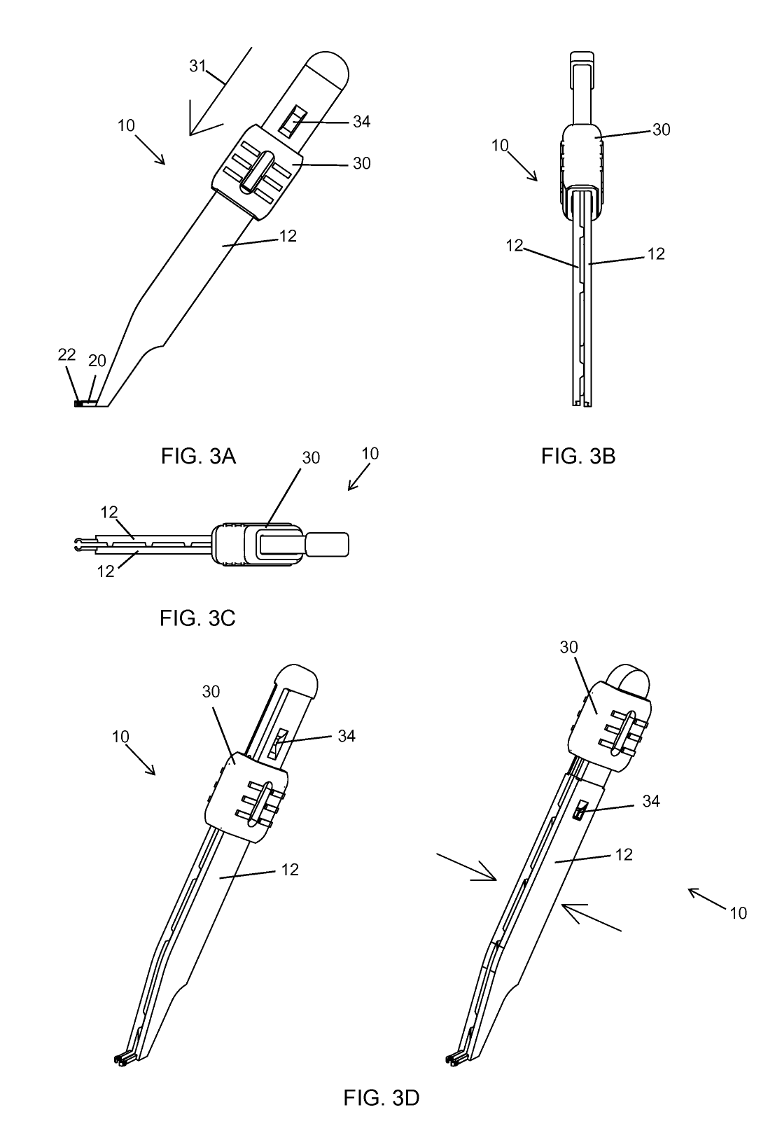

Referring to FIGS. 3A-3C, iris retractor forceps 10 is in a contracted (squeezed, gripping) orientation, in which the forceps legs 12 are squeezed together. The locking element 30 is slid distally (in the direction of arrow 31) so that flexible member 38 of locking element 30 is locked into the distal groove 34 (not seen in FIGS. 3A-3C). It is noted that one or more additional grooves may be provided between grooves 32 and 34, so that the forceps 10 has more than one locking position, such as a fully squeezed position and a partially squeezed position. In another embodiment, locking position is achieved by providing one or more friction/locking means. In some embodiment, the term “groove” is interchangeble with the term “elongate channel”, “channel”, or “elongated channel”. Reference is now made to FIGS. 3D-3F, iris retractor forceps 10 is in a contracted (squeezed, gripping) orientation, in which the forceps legs 12 are squeezed together. The locking element 30 is not slid distally, but the forceps are contracted and held by the user's fingers.

Reference is now made to FIGS. 4A-4D. A holder 40 is provided to hold an iris retractor assembly 42, similar to iris retractor assemblies 70 and 90 as disclosed in WO 2013/062983. The holder 40 greatly simplifies handling, manipulation and loading of the iris retractor 42 in preparation for the surgical procedure. A similar holder is also shown in PCT Patent Application PCT/US2012/061513 of the present assignee.

In the illustrated embodiment, holder 40 may include a base 44 on which one or more iris retractor assemblies 46 (typically a pair of iris retractors) may be positioned.

Holding assembly 46 includes two blocks 48, each formed with a groove 50. When the iris retractor 42 is held in iris retractor holding assembly 46, its slender elements 43 rest against opposing grooves 50, as seen in FIGS. 4B and 4D. The proximal portion of iris retractor 42 is received in a recess 52 formed on base 44. Iris retractor forceps 10 can easily grasp iris retractor 42, for example, while it is held in holder 40. When positioning protrusion 20 of iris retractor forceps 10 on surface 49 of holder 40 and contracting the his retractor forceps 10, curved grasping tips 22 are guided to easily engage and grasp iris retractor 42.

The locking element 30 enables forceps 10 to hold a loaded iris retractor, without the need of holding the forceps contracted by hand, such as when the retractor is not yet in use or when passing the retractor from one person to another, or when the retractor is loaded by the assistant for postponed use. Loaded forceps may be left loaded and locked on the sterile table for postponed physician's use

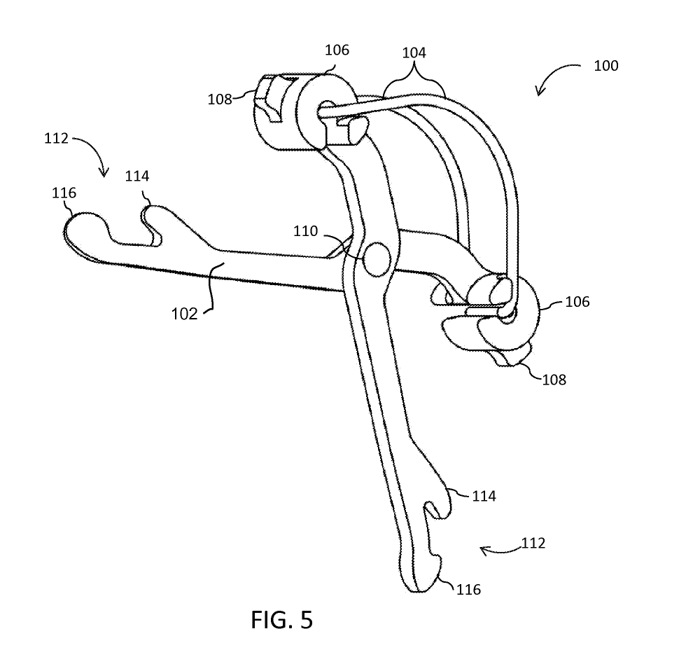

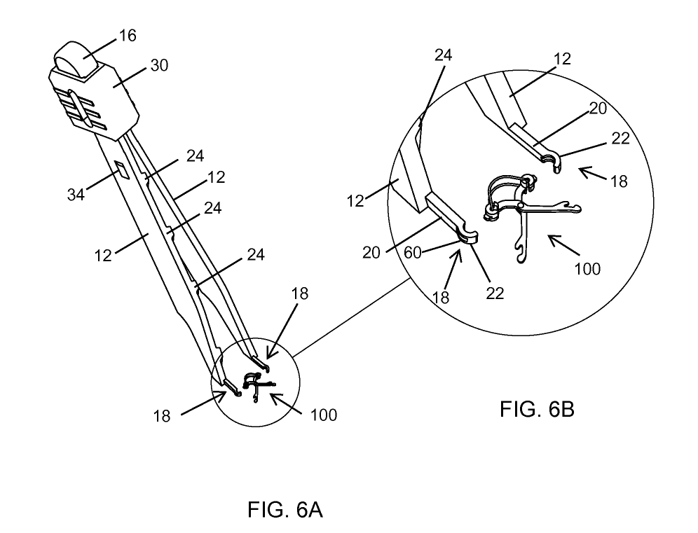

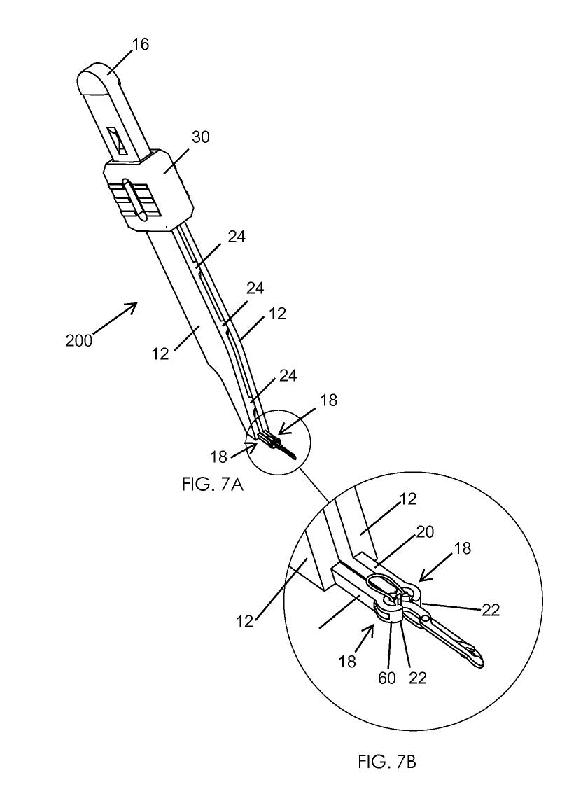

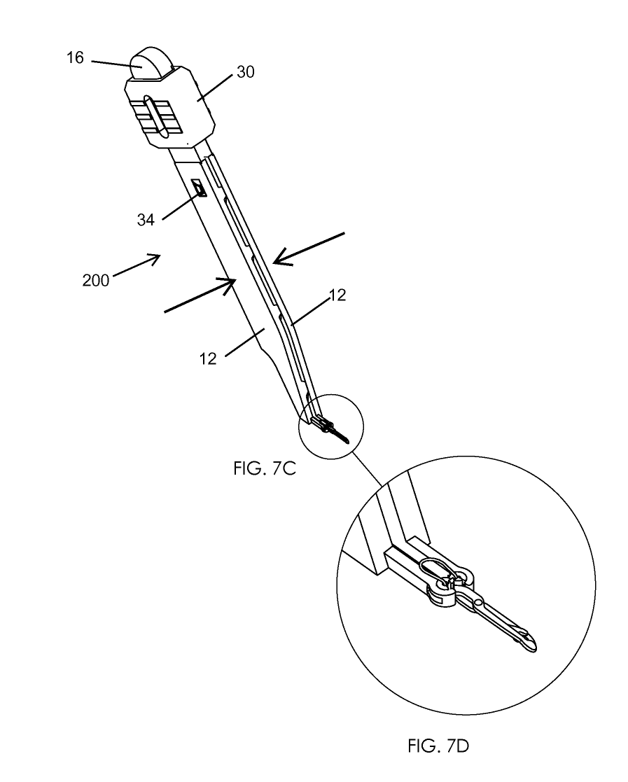

Forceps 10 may be adapted to grasp iris retractor assemblies by utilizing grasping mechanisms as disclosed in WO 2013/062983. An exemplary mechanism for holding an exemplary iris retractor, as shown in FIG. 5, is shown in FIGS. 6A-7B.

In some embodiment, a pair of iris retractors as described herein are used in kits and in medical procedures as described herein. In another embodiment, the forceps of the invention are adapted to grasp a single iris retractor in a non-expanded orientation (i.e., retracted position) and then used for placing each iris retractor in the eye. In another embodiment, adapted to grasp includes a structure for securing an iris retractor within the forceps which allows the insertion of an iris retractor in a retracted position through a small incision in the limbus. In another embodiment, the small incision is 0.5 to 2.5 mm wide. In another embodiment, the small incision is 0.7 to 1.8 mm wide. In another embodiment, the small incision is 0.8 to 1.6 mm wide. In another embodiment, the small incision is 1.0 to 1.5 mm wide.

In another embodiment, the forceps and a pair of iris retractors are utilized in various ophthalmic procedures that require the dilation of the pupil. For example, a lens with a cataract is typically removed from the eye by phacoemulsification. In another embodiment, a pair of iris retractors are inserted into an affected eye with the forceps thus extending the pupil during phacoemulsification thereby providing the surgeon with a wide view of the lens. In another embodiment, extending the pupil is retracting the iris with what is referred to as an iris retractor, and holding the iris at its outer edges.

In another embodiment, the iris retractor forceps (also referred to as forceps) specifically fit, grasp and hold a single iris retractor assembly via the retractor's handles (two handles for each iris retractor) or proximal forceps grasping means. In another embodiment, the forceps pull out a single iris retractor from its housing. In another embodiment, the forceps are used to pull out a single iris retractor from a holding assembly comprising or consisting at least one iris retractor assembly. In another embodiment, the forceps are used to pull out a single iris retractor from a holding assembly comprising or consisting two iris retractor assemblies. In another embodiment, the forceps are used to pull out a single iris retractor from a holding assembly. In another embodiment, the iris retractor is the iris retractor described in PCT Patent Application Publication WO 2013/062983 which is hereby incorporated by reference in its entirety. In another embodiment, the iris retractor is secured or semi-secured to the forceps via one or more grasping tips located at the proximal ends of each handle of the iris retractor. In another embodiment, the iris retractor is secured or semi-secured to the forceps via two two protruding ears wherein each protruding ear is disposed at the proximal end of each one the two slender elements. In another embodiment, a holder (such as a housing) comprises one or more iris retractor holding assemblies wherein each iris retractor holding assembly is configured to hold one or more iris retractor assemblies. In another embodiment, a holder (such as a housing) comprises one or more iris retractor holding assemblies wherein each iris retractor holding assembly is configured to hold one iris retractor. In another embodiment, the holder (such as a housing) and iris retractor holding assembly maintain the one or more iris retractors, sterile. In another embodiment, the holder (such as a housing) and iris retractor holding assembly protect the one or more iris retractors from damage such as but not limited to physical damage, contamination, humidity, or any combination thereof.

FIG. 5 is an illustrations of simplified perspective view of an exemplary iris retractor assembly 100 in an expanded orientation, in accordance with an embodiment. Iris retractor 100 may include two slender elements 102, a double biasing member 104, proximal ends 106, protruding ears 108, a pivot member 110 and iris grabbing hooks 112. Each of iris grabbing hook 112 may include a proximal tooth 114 and a distal tooth 116.

Proximal ends 106 may be disposed or formed at a proximal end of one or more slender elements 102. Iris grabbing hooks 112 may be disposed or formed at a distal end of one or more slender elements 102. Slender elements 102 may be pivoted about pivot member 110. Protruding ears 108 may be disposed or formed at a proximal end of one or more slender elements 102 and at edges of proximal ends 106.

Slender elements 102 may be biased against each other by biasing member 104, such as a springy, resilient wire, band, leaf spring and the like, connected to proximal ends 106 of slender elements 102.

In usage, iris tissue may be held between distal tooth 116 and proximal tooth 114, wherein distal tooth 116 may be below the iris and proximal tooth 114 may be above the iris. In another embodiment, usage, iris tissue may be held between distal tooth 116 and proximal tooth 114, wherein proximal tooth 114 may be below the iris and distal tooth 116 may be above the iris.

Optionally, distal tooth 116 may curve outwardly (outwardly in the sense of outwards from an imaginary centerline that passes through pivot member 100 and the middle of biasing member 104) and proximal tooth 114 may be tilted towards distal tooth 116, as shown in the illustrated embodiment of FIG. 5. Distal tooth 116 and/or proximal tooth 114 may have rounded ends. Optionally, distal tooth 116 may curve downwardly.

In some embodiments, distal tooth 116 may be a rounded protuberance, wider than the width of slender element 102. Proximal tooth 114 may curve slightly outwardly.

FIGS. 6A and 6B are illustrations of simplified perspective and perspective enlarged views of an exemplary his retractor forceps in an expanded orientation and iris retractor assembly 100 of FIG. 5. FIGS. 7A and 7B are illustrations of simplified perspective and perspective enlarged views of iris retractor forceps of FIGS. 6A and 6B in a contracted orientation with locking element 30 slid distally holding iris retractor assembly 100 of FIG. 5 in a contracted orientation.

The double biasing member 104 may provide a balanced and centered (symmetric) spring force on slender elements 102 of iris retractor 100. In other words, the spring force may be symmetric about pivot member 110 and this may prevent retractor 100 from twisting or bending due to asymmetric spring forces. For example, this may enable use of plastics to make retractor 100, because of the reduced or negligible spring forces on slender elements 102. In some embodiments, a biasing member which includes only one biasing element, as opposed to double biasing member 104, may be used.

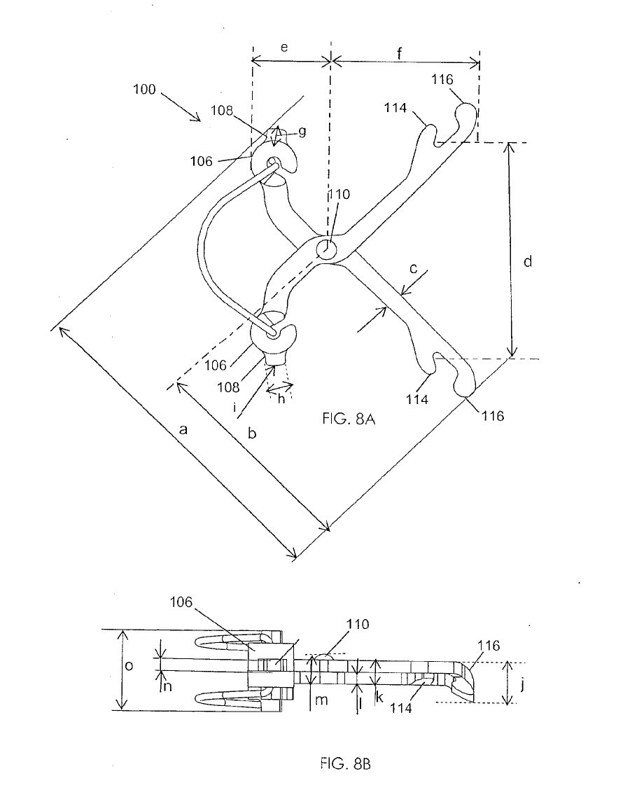

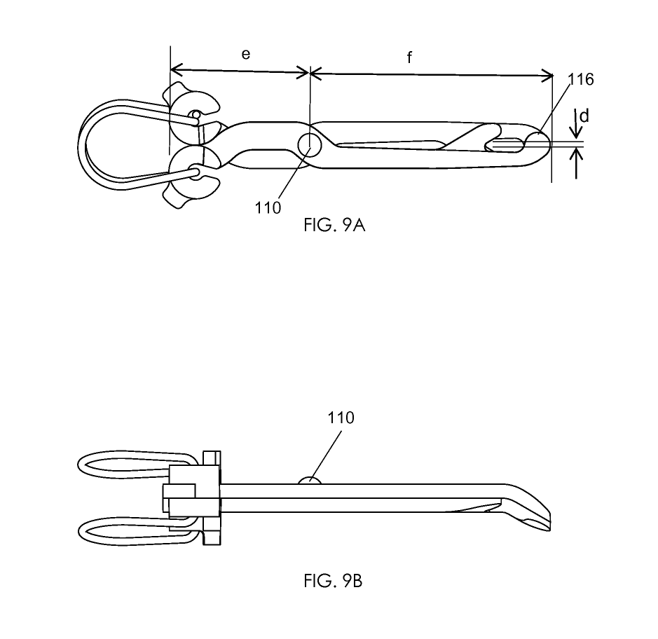

Reference is now made to FIG. 8A, which is an illustration of a top view of the iris retractor assembly of FIG. 5 in an extended orientation, indicating dimensions of the iris retractor assembly, as described herein below.

In another embodiment, the phrases “iris retractor” and “iris retractor assembly” are used interchangebly.

In some embodiments, “a” is the maximal length of slender element 102 of iris retractor 100 (i.e., from protruding ear 108 to distal tooth 116). In some embodiments, “a” equals to 4.05 to 12.3 millimeters (mm). In some embodiments, “a” equals to 5.5 to 11 mm. In some embodiments, “a” equals to 7 to 9.5 mm. In some embodiments, “a” equals to 8 to 8.8 mm. In some embodiments, “a” equals to 8.1 to 8.3 mm. In some embodiments, “b” is the maximal length of slender element 102 of iris retractor 100 from pivot member 110 to distal tooth 116. In some embodiments, “b” equals to 2.55 to 7.8 mm. In some embodiments, “b” equals to 3 to 7 mm. In some embodiments, “b” equals to 3.5 to 6.5 mm. In some embodiments, “b” equals to 4.3 to 5.8 mm. In some embodiments, “b” equals to 5 to 5.4 mm.

In some embodiments, “c” is the minimal width of a portion of slender element 102 of iris retractor 100 extending from pivot member 110 to proximal tooth 114. In some embodiments, “c” equals to 0.19 to 0.63 mm. In some embodiments, “c” equals to 0.24 to 0.58 mm. In some embodiments, “c” equals to 0.29 to 0.53 mm. In some embodiments, “c” equals to 0.34 to 0.48 mm. In some embodiments, “c” equals to 0.38 to 0.43 mm.

In some embodiments, “d” is the distance between the inner edges of proximal teeth 114 of iris retractor 100. In some embodiments, “d”, when slender element 102 is in a maximal extended orientation, equals to 2.9 to 9.3 mm. In some embodiments, “d”, when slender element 102 is in a maximal extended orientation equals to 3.6 to 8.5 mm. In some embodiments, “d”, when slender element 102 is in a maximal extended orientation equals to 4.3 to 7.7 mm. In some embodiments, “d”, when slender element 102 is in a maximal extended orientation equals to 5.05 to 6.95 mm. In some embodiments, “d”, when slender element 102 is in a maximal extended orientation equals to 5.8 to 6.2 mm.

In some embodiments, “d”, when slender element 102 is in a maximal contracted orientation equals to 0.07 to 0.38 mm. In some embodiments, “d”, when slender element 102 is in a maximal contracted orientation equals to 0.11 to 0.34 mm. In some embodiments, “d”, when slender element 102 is in a maximal contracted orientation equals to 0.14 to 0.3 mm. In some embodiments, “d”, when slender element 102 is in a maximal contracted orientation equals to 0.16 to 0.27 mm. In some embodiments, “d”, when slender element 102 is in a maximal contracted orientation equals to 0.18 to 0.23 mm.

In some embodiments, “e” is the distance between a proximal edge of proximal end 106 and pivot member 110 of iris retractor 100. In some embodiments, “e”, when slender element 102 is in a maximal extended orientation, equals to 0.45 to 1.44 mm. In some embodiments, “e”, when slender element 102 is in a maximal extended orientation, equals to 0.55 to 1.34 mm. In some embodiments, “e”, when slender element 102 is in a maximal extended orientation, equals to 0.65 to 1.25 mm. In some embodiments, “e”, when slender element 102 is in a maximal extended orientation, equals to 0.75 to 1.15 mm. In some embodiments, “e”, when slender element 102 is in a maximal extended orientation, equals to 0.85 to 1.04 mm.

In some embodiments, “e”, when slender element 102 is in a maximal contracted orientation, equals to 1.47 to 4.56 mm. In some embodiments, “e”, when slender element 102 is in a maximal contracted orientation, equals to 1.87 to 4.15 mm. In some embodiments, “e”, when slender element 102 is in a maximal contracted orientation, equals to 2.2 to 3.75 mm. In some embodiments, “e”, when slender element 102 is in a maximal contracted orientation, equals to 2.6 to 3.4 mm. In some embodiments, “e”, when slender element 102 is in a maximal contracted orientation, equals to 2.8 to 3.3 mm. In some embodiments, “f” is the distance between pivot member 110 and a distal edge of distal tooth 116 of iris retractor 100. In some embodiments, “f” equals to 1.5 to 4.74 mm when slender element 102 is in a maximal extended orientation. In some embodiments, “f” equals to 1.9 to 4.3 mm when slender element 102 is in a maximal extended orientation. In some embodiments, “f” equals to 2.3 to 3 9 mm when slender element 102 is in a maximal extended orientation. In some embodiments, “f” equals to 2.7 to 3 5 mm when slender element 102 is in a maximal extended orientation. In some embodiments, “f” equals to 2.85 to 3.3 mm when slender element 102 is in a maximal extended orientation.

In some embodiments, “f” equals to 2.55 to 7 8 mm when slender element 102 is in a maximal contracted orientation. In some embodiments, “f” equals to 3.15 to 7 2 mm when slender element 102 is in a maximal contracted orientation. In some embodiments, “f” equals to 3.75 to 6.6 mm when slender element 102 is in a maximal contracted orientation. In some embodiments, “f” equals to 4.4 to 6 mm when slender element 102 is in a maximal contracted orientation. In some embodiments, “f” equals to 4.8 to 5 4 mm when slender element 102 is in a maximal contracted orientation.

In some embodiments, “g” is the maximal buldge of protruding ear 108 of iris retractor 100. In some embodiments, “g” equals to 0.13 to 0.5 mm. In some embodiments, “g” equals to 0.18 to 0.45 mm. In some embodiments, “g” equals to 0.23 to 0.4 mm. In some embodiments, “g” equals to 0.26 to 0.37 mm. In some embodiments, “g” equals to 0.28 to 0.33 mm.

In some embodiments, “h” is the maximal length of protruding ear 108 of iris retractor 100. In some embodiments, “h” equals to 0.22 to 0.83 mm. In some embodiments, “h” equals to 0.27 to 0.77 mm. In some embodiments, “h” equals to 0.32 to 0.71 mm. In some embodiments, “h” equals to 0.4 to 0.66 mm. In some embodiments, “h” equals to 0.45 to 0.58 mm. In some embodiments, “i” is the radius of Protruding ears 108. In some embodiments, “i” is 0.7 to 1.2 mm. In some embodiments, “i” is 0.8 to 1.1 mm. In some embodiments, “i” is 0.8 to 1.0 mm. In some embodiments, “i” is 0.86 to 0.9 mm.

Reference is now made to FIG. 8B, which is an illustration of a side view of the iris retractor assembly of FIG. 8A, indicating dimensions of the iris retractor assembly, as described herein below.

In some embodiments, “j” is the depth or thickness of two slender elements 102 of retractor 100, as shown in FIG. 8B. In some embodiments, “j” equals to 0.45 to 1.65 mm. In some embodiments, “j” equals to 0.6 to 1.5 mm. In some embodiments, “j” equals to 0.75 to 1.35 mm. In some embodiments, “j” equals to 0.85 to 1.2 mm. In some embodiments, “j” equals to 0.95 to 1.05 mm.

In some embodiments, “k” is the maximal depth or thickness of a middle portion of two slender elements 102 of retractor 100, as shown in FIG. 8B. In some embodiments, “k” equals to 0.26 to 1.01 mm. In some embodiments, “k” equals to 0.36 to 0.92 mm. In some embodiments, “k” equals to 0.46 to 0.82 mm. In some embodiments, “k” equals to 0.5 to 0.75 mm. In some embodiments, “k” equals to 0.58 to 0.65 mm.

In some embodiments, “l” is the maximal depth or thickness of the upper slender element 102 (i.e., as held by a medical professional with respect to a treated iris) of two slender elements 102 of retractor 100, as shown in FIG. 8B. In some embodiments, “l” equals to 0.14 to 0.48 mm. In some embodiments, “l” equals to 0.17 to 0.45 mm. In some embodiments, “l” equals to 0.2 to 0.42 mm. In some embodiments, “l” equals to 0.23 to 0.38 mm. In some embodiments, “l” equals to 0.25 to 0.32 mm.

In some embodiments, “m” is a depth or thickness of a middle portion of two slender elements 102 and the pivot member 110 heat formed top to connect the slender elements to each other of retractor 100, as shown in FIG. 8B. In some embodiments, “m” equals to 0.32 to 1.28 mm. In some embodiments, “m” equals to 0.4 to 1.18 mm. In some embodiments, “m” equals to 0.5 to 1.08 mm. In some embodiments, “m” equals to 0.6 to 0.95 mm. In some embodiments, “m” equals to 0.7 to 0.8 mm.

In some embodiments, “n” is the maximal depth or thickness of protruding ear 108 of retractor 100, as shown in FIG. 8B. In some embodiments, “n” equals to 0.01 to 0.5 mm. In some embodiments, “n” equals to 0.06 to 0.45 mm. In some embodiments, “n” equals to 0.11 to 0.4 mm. In some embodiments, “n” equals to 0.2 to 0.35 mm. In some embodiments, “n” equals to 0.28 to 0.32 mm.

In some embodiments, “o” equals to 0.8 to 2.7 mm. In some embodiments, “o” equals to 1.05 to 2.45 mm. In some embodiments, “o” equals to 1.3 to 2.2 mm. In some embodiments, “o” equals to 1.55 to 1.95 mm. In some embodiments, “o” equals to 1.65 to 1.75 mm.

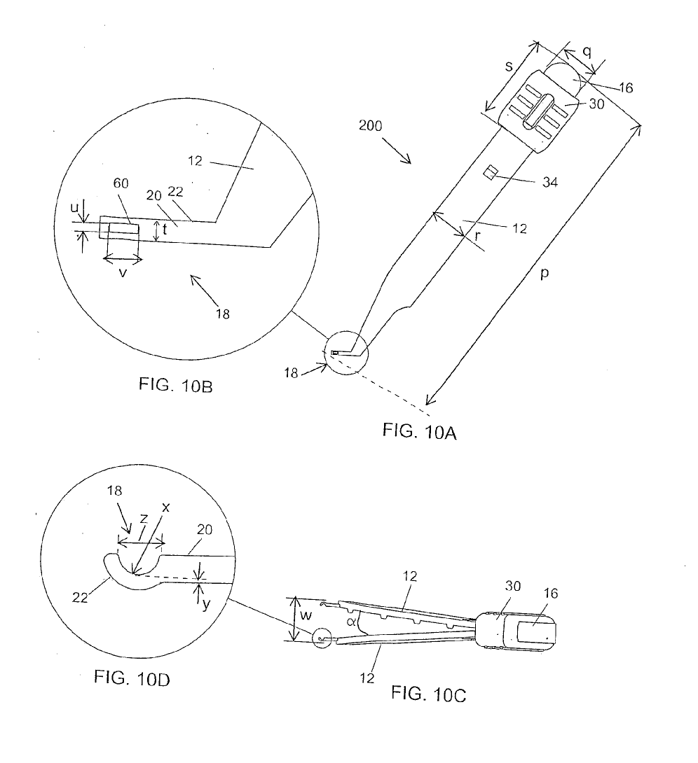

Reference is now made to FIG. 10A, which is an illustration of a side view of the iris retractor forceps of FIG. 1 A, indicating dimensions of the iris retractor forceps, as described herein below.

In some embodiments, “p” is the length of the forceps. In some embodiments, “p” is between 60 to 200 mm. In some embodiments, “p” is between 80 to 150 mm. In some embodiments, “p” is between 90 to 120 mm. In some embodiments, “p” is between 110 to 115 mm. In some embodiments, “p” is between 112 to 113 mm.

In some embodiments, “q” is the maximal width of locking element 30. In some embodiments, “q” equals to 0.45 to 1.65 mm. In some embodiments, “q” equals to 0.6 to 1.5 mm. In some embodiments, “q” equals to 0.75 to 1.35 mm. In some embodiments, “q” equals to 0.85 to 1.2 mm. In some embodiments, “q” equals to 0.95 to 1.05 mm.

In some embodiments, “r” is the maximal width of forceps leg 12. In some embodiments, “r” equals to 0.26 to 1.01 mm. In some embodiments, “r” equals to 0.36 to 0.92 mm. In some embodiments, “r” equals to 0.46 to 0.82 mm. In some embodiments, “r” equals to 0.5 to 0.75 mm. In some embodiments, “r” equals to 0.58 to 1.65 mm.

In some embodiments, “s” is the maximal total length of locking element 30 and handle member 16. In some embodiments, “s” equals to 0.14 to 0.48 mm. In some embodiments, “s” equals to 0.17 to 0.45 mm. In some embodiments, “s” equals to 0.2 to 0.42 mm. In some embodiments, “s” equals to 0.23 to 0.38 mm. In some embodiments, “s” equals to 0.25 to 0.32 mm.

Reference is now made to FIG. 10B, which is an illustration of an enlarged side view of the indicated portion of the iris retractor forceps of FIG. 10A, indicating dimensions of the iris retractor forceps, as described herein below. In some embodiments, “t” is the maximal width of distal curved grasping tip 22. In some embodiments, “t” equals to 0.32 to 1.28 mm. In some embodiments, “t” equals to 0.4 to 1.18 mm. In some embodiments, “t” equals to 0.5 to 1.08 mm. In some embodiments, “t” equals to 0.6 to 0.95 mm. In some embodiments, “t” equals to 0.7 to 0.8 mm.

In some embodiments, “u” is the maximal width of notch 60. In some embodiments, “u” equals to 0.01 to 0.5 mm. In some embodiments, “u” equals to 0.06 to 0.45 mm. In some embodiments, “u” equals to 0.11 to 0.4 mm. In some embodiments, “u” equals to 0.2 to 0.35 mm. In some embodiments, “u” equals to 0.28 to 0.32 mm.

In some embodiments, “v” is the maximal length of distal curved grasping tip 22. In some embodiments, “v” equals to 0.8 to 2.7 mm. In some embodiments, “v” equals to 1.05 to 2.45 mm. In some embodiments, “v” equals to 1.3 to 2.2 mm. In some embodiments, “v” equals to 1.55 to 1.95 mm. In some embodiments, “v” equals to 1.65 to 1.75 mm.

Reference is now made to FIG. 10C, which is an illustration of a top view of the iris retractor forceps of FIG. 1A, indicating dimensions of the iris retractor forceps, as described herein below.

In some embodiments, “w” is the distance between distal graspers 18. In some embodiments, “w” equals to 56 to 169.5 mm. In some embodiments, “w” equals to 70 to 165 mm when graspers 18 are in a maximal extended orientation. In some embodiments, “w” equals to 85 to 160 mm when graspers 18 are in a maximal extended orientation. In some embodiments, “w” equals to 95 to 145 mm when graspers 18 are in a maximal extended orientation. In some embodiments, “w” equals to 105 to 130 mm when graspers 18 are in a maximal extended orientation.

In some embodiments, “w” equals to 1 to 18 mm. In some embodiments, “w” equals to 2 to 5 mm when graspers 18 are in a maximal contracted orientation. In some embodiments, “w” equals to 2.5 to 4 5 mm when graspers 18 are in a maximal contracted orientation. In some embodiments, “w” equals to 2.2 to 4 mm when graspers 18 are in a maximal contracted orientation. In some embodiments, “w” equals to 8 to 18 mm when graspers 18 are in a maximal expanded orientation. In some embodiments, “w” equals to 10 to 16 mm when graspers 18 are in a maximal expanded orientation.

In some embodiments, “α” (or alpha) is the angle between forceps legs 12. In some embodiments, “α”(or alpha) equals to 5.75 to 17.55 degrees when forceps legs 12 are in a maximal extended orientation. In some embodiments, “α” (or alpha) equals to 7.25 to 16 degrees when forceps legs 12 are in a maximal extended orientation. In some embodiments, “α” (or alpha) equals to 8.75 to 14.5 degrees when forceps legs 12 are in a maximal extended orientation. In some embodiments, “α” (or alpha) equals to 10.25 to 13 degrees when forceps legs 12 are in a maximal extended orientation.

In some embodiments, measures/dimensions for the devices of the invention are further provided in table 1:

TABLE 1
(dimensions are in mm; only angles such as “alpha” are in degrees)

| | Dimension markings | | | | | | | | |
|---|---|---|---|---|---|---|---|---|---|
| | a | | | b | | | c | | |
| Configuration | nominal | min | max | nominal | min | max | nominal | min | max |
| Free | 8.16 | 7.8 | 8.6 | 5.15 | 4.6 | 5.7 | 0.4 | 0.3 | 0.5 |
| Firmly closed | 8.16 | 7.8 | 8.6 | 5.15 | 4.6 | 5.7 | 0.4 | 0.3 | 0.5 |
| | Dimension markings | | | | | | | | |
| | d | | | e | | | f | | |
| Configuration | nominal | min | max | nominal | min | max | nominal | min | max |
| Free | 6 | 5.0 | 7.2 | 0.93 | 0.80 | 1.2 | 3.08 | 2.6 | 3.32 |
| Firmly closed | 0.2 | 0.1 | 0.3 | 3 | 2.5 | 3.5 | 5.15 | 4.8 | 5.5 |
| | Dimension markings | | | | | | | | |
| | g | | | h | | | i (radius, mm) | | |
| Configuration | nominal | min | max | nominal | min | max | nominal | min | max |
| Free | 0.3 | 0.2 | 0.4 | 0.5 | 0.4 | 0.6 | 0.875 | 0.8 | 0.95 |
| Firmly closed | 0.3 | 0.2 | 0.4 | 0.5 | 0.4 | 0.6 | 0.875 | 0.8 | 0.95 |
| | Dimension markings | | | | | | | | |
| | j | | | k | | | l | | |
| Configuration | nominal | min | max | nominal | min | max | nominal | min | max |
| Open | 1 | 0.7 | 1.3 | 0.6 | 0.45 | 0.75 | 0.3 | 0.2 | 0.4 |
| Closed | 1 | 0.7 | 1.3 | 0.6 | 0.45 | 0.75 | 0.3 | 0.2 | 0.4 |
| | Dimension markings | | | | | | | | |
| | m | | | n | | | o | | |
| Configuration | nominal | min | max | nominal | min | max | nominal | min | max |
| Open | 0.75 | 0.6 | 0.95 | 0.3 | 0.2 | 0.4 | 1.7 | 1.4 | 2.1 |
| Closed | 0.75 | 0.6 | 0.9 | 0.3 | 0.2 | 0.4 | 1.7 | 1.4 | 2.1 |

| | Dimension markings | | | | | | | | | | | |
|---|---|---|---|---|---|---|---|---|---|---|---|---|
| | s | | | t | | | u | | | v | | |
| Configuration | nominal | min | max | nominal | min | max | nominal | min | max | nominal | min | max |
| Open | 11.6 | 11.2 | 12.1 | 1.1 | 0.80 | 1.3 | 0.4 | 0.2 | 0.7 | 1.38 | 1.1 | 1.7 |
| Closed | 11.6 | 11.2 | 12.1 | 1.1 | 0.80 | 1.3 | 0.4 | 0.2 | 0.7 | 1.38 | 1.1 | 1.7 |

| | Dimension markings | | | | | | | | |
|---|---|---|---|---|---|---|---|---|---|
| | p | | | q | | | r | | |
| Configuration | nominal | min | max | nominal | min | max | nominal | min | max |
| Free | 112.5 | 80 | 145 | 11.6 | 8.0 | 15.0 | 27 | 22 | 31 |
| Firmly closed | 112.5 | 80 | 145 | 11.6 | 8.0 | 15.0 | 46.5 | 41 | 52 |

| | Dimension markings | | | | | | | | | | | |
|---|---|---|---|---|---|---|---|---|---|---|---|---|
| | a[deg] | | | w | | | x (diameter, mm) | | | y | | |
| Configuration | nominal | min | max | nominal | min | max | nominal | min | max | nominal | min | max |
| Open | 6.2 | 5.2 | 7.4 | 11.6 | 10.2 | 13.2 | 1.2 | 0.7 | 1.7 | 0.25 | 0.1 | 0.5 |
| Closed | 0 | 0 | 1 | 3.2 | 1.8 | 4.6 | 1.2 | 0.7 | 1.7 | 0.25 | 0.1 | 0.5 |

| | Dimension markings | | |
|---|---|---|---|
| | z | | |
| Configuration | nominal | min | max |
| Open | 1.2 | 0.8 | 1.8 |
| Closed | 1.2 | 0.8 | 1.8 |

Reference is now made to FIG. 10D, which is an illustration of an enlarged top view of the indicated portion of the iris retractor forceps of FIG. 10C, indicating dimensions of the iris retractor forceps, as described herein below.

In some embodiments, “x” is the maximal diameter of a circle formed by the curve of distal curved grasping tip 22 of forceps, as shown in FIG. 10D. In some embodiments, “x” equals to 0.58 to 1.85 mm. In some embodiments, “x” equals to 0.7 to 1.7 mm. In some embodiments, “x” equals to 0.85 to 1.55 mm. In some embodiments, “x” equals to 1 to 1.4 mm. In some embodiments, “x” equals to 1.1 to 1.3 mm.

In some embodiments, “y” is the maximal depth of distal curved grasping tip 22 of forceps, as shown in FIG. 10D. In some embodiments, “y” equals to 0.1 to 0.45 mm. In some embodiments, “y” equals to 0.12 to 0.42 mm. In some embodiments, “y” equals to 0.15 to 0.4 mm. In some embodiments, “y” equals to 0.18 to 0.35 mm. In some embodiments, “y” equals to 0.21 to 0.3 mm.

In some embodiments, “z” is the minimal distance between the two ends of distal curved grasping tip 22 of forceps, as shown in FIG. 10D. In some embodiments, “z” equals to 0.57 to 1.88 mm. In some embodiments, “z” equals to 0.72 to 1.73 mm. In some embodiments, “z” equals to 0.87 to 1.58 mm. In some embodiments, “z” equals to 1.02 to 1.4 mm. In some embodiments, “z” equals to 1.15 to 1.25 mm.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the market site, or to enable others of ordinary skill in the art to understand the embodiments disclosed herein.

What is claimed is:

1. An assembly comprising:
an iris retractor forceps comprising two forceps legs which join together at a proximal junction, at least two grooves, said forceps legs comprising distal graspers for grasping an iris retractor or a holder for an iris retractor; and
a locking element slidingly received in a groove formed in said forceps legs, said locking element sliding between at least two positions, said at least two positions comprising:
(a) a first position in which the forceps legs are spread apart, and
(b) a second position in which the forceps legs are squeezed together,
wherein in the second position said locking element is held in a locked position within said groove.

2. The assembly according to claim 1, wherein said at least two grooves comprise one proximal and one distal, and said locking element comprises a bulging flexible member that locks into said at least two grooves.

3. The assembly according to claim 1, wherein each of said distal graspers comprises a protrusion that angularly extends from a corresponding forceps leg of said forceps legs.

4. The assembly according to claim 3, wherein said protrusion comprises a distal, curved grasping tip.

5. The assembly according to claim 1, wherein one or more stoppers are provided on inner surfaces of said forceps legs.

6. A kit comprising:
one or more iris retractor assemblies;
a holder comprising one or more iris retractor holding assemblies configured to hold said one or more iris retractor assemblies;
an iris retractor forceps comprising two forceps legs which join together at a proximal junction, said forceps legs comprising distal graspers for grasping the holder; and
a locking element slidingly received in a groove formed in said forceps legs, said locking element sliding between at least two positions, said at least two positions comprising:
(a) a first position in which the forceps legs are spread apart, and
(b) a second position in which the forceps legs are squeezed together,
wherein in the second position said locking element is held in a locked position.

7. The kit of claim 6, wherein:
each of said one or more iris retractor holding assemblies comprises two blocks, each block of said two blocks formed with a groove,
and wherein when said one or more iris retractor assemblies are held in said holder, said slender elements rest against said two grooves.

8. The kit of claim 6, wherein said holder comprises a base, said base comprising a recess, and wherein when an iris retractor assembly of said one or more iris retractor assemblies is held in said holder, a proximal portion of said iris retractor is received in said recess.

9. A kit comprising:
one or more iris retractor assemblies;
an iris retractor forceps comprising two forceps legs which join together at a proximal junction, said forceps legs comprising distal curved grasping tips for grasping an iris retractor assembly of said one or more iris retractor assemblies; and
a locking element slidingly received in a groove formed in said forceps legs, said locking element sliding between at least two positions, said at least two positions comprising:
(a) a first position in which the forceps legs are spread apart, and
(b) a second position in which the forceps legs are squeezed together,
wherein in the second position said locking element is held in a locked position.

10. The kit of claim 9, wherein the one or more iris retractor assemblies comprise:
(a) two slender elements, and
(b) two protruding ears disposed at proximal ends of the two slender elements,
and wherein the distal curved grasping tips comprise notches configured to receive the protruding ears.
